(19) United States
(12) Patent Application Publication
(10) Pub. No.: US 2012/0108962 A1
(43) Pub. Date: May 3, 2012

(54) PROVIDING A BODY MARK IN AN ULTRASOUND SYSTEM

(75) Inventor: Sook Gon YOON, Seoul (KR)

(73) Assignee: SAMSUNG MEDISON CO., LTD.

(21) Appl. No.: 13/276,705

(22) Filed: Oct. 19, 2011

(30) Foreign Application Priority Data
Nov. 2, 2010 (KR) 10-2010-0108249

(51) Int. Cl. A61B 8/00

(52) U.S. Cl. 600/437

(57) ABSTRACT

Embodiments for providing a body mark are disclosed. In one embodiment, by way of non-limiting example, an ultrasound system comprises: an ultrasound data acquisition unit configured to acquire ultrasound data corresponding to a living body including a target object; a storage unit for storing at least one body mark corresponding to at least one application; and a processing unit configured to form an ultrasound image based on the ultrasound data, perform edge detection upon the ultrasound image to detect an edge of the target object, detect a body mark corresponding to the edge from the storage unit based on first input information for selecting an application, and set the body mark on the ultrasound image based on the edge.

### CROSS-REFERENCE TO RELATED APPLICATION

The present application claims priority from Korean Patent Application No. 10-2010-0108249 filed on Nov. 2, 2010, the entire subject matter of which is incorporated herein by reference.

### TECHNICAL FIELD

The present disclosure generally relates to ultrasound systems, and more particularly to providing a body mark in an ultrasound system.

### BACKGROUND

An ultrasound system has become an important and popular diagnostic tool since it has a wide range of applications. Specifically, due to its non-invasive and non-destructive nature, the ultrasound system has been extensively used in the medical profession. Modern high-performance ultrasound systems and techniques are commonly used to produce two-dimensional or three-dimensional ultrasound images of internal features of a target object (e.g., human organs).

The ultrasound system may transmit ultrasound signals to a living body, which includes a target object (e.g., a heart, a fetus, etc.), and receive ultrasound signals (i.e., ultrasound echo signals) from the living body to thereby form a 2D (two-dimensional) ultrasound image or a 3D (three-dimensional) ultrasound image. The 2D or 3D ultrasound image may be displayed on a display unit.

The ultrasound system may provide the ultrasound image and a body mark, which represents the target object as a picture or icon, to improve the convenience of a user and patient. The ultrasound system may set the body mark selected by the user on the ultrasound image. However, there is a problem since a different body mark from the target object may be provided. Also, the ultrasound system may set the body mark on a part of the ultrasound image. However, there is a problem that a position relationship between the body mark and the ultrasound image may not be provided clearly.

### SUMMARY

Embodiments for providing a body mark corresponding to an application on an ultrasound image based on at least one body mark corresponding to at least one body mark in an ultrasound system are disclosed herein.

In one embodiment, by way of non-limiting example, an ultrasound system comprises: an ultrasound data acquisition unit configured to acquire ultrasound data corresponding to a living body including a target object; a storage unit for storing at least one body mark corresponding to at least one application; and a processing unit configured to form an ultrasound image based on the ultrasound data, perform edge detection upon the ultrasound image to detect an edge of the target object, detect a body mark corresponding to the edge from the storage unit based on first input information for selecting an application, and set the body mark on the ultrasound image based on the edge.

In another embodiment, there is a method of providing a body mark, comprising: a) acquiring ultrasound data corresponding to a living body including a target object; b) forming an ultrasound image based on the ultrasound data; c) performing edge detection upon the ultrasound image to detect an edge of the target object; d) detecting a body mark corresponding to the edge from a storage unit for storing at least one body mark corresponding to at least one application based on first input information for selecting an application; and e) setting the body mark on the ultrasound image based on the edge.

In yet another embodiment, there is provided a computer readable medium comprising computer executable instructions configured to perform the following acts: a) acquiring ultrasound data corresponding to a living body including a target object; b) forming an ultrasound image based on the ultrasound data; c) performing edge detection upon the ultrasound image to detect an edge of the target object; d) detecting a body mark corresponding to the edge from a storage unit for storing at least one body mark corresponding to at least one application based on first input information for selecting an application; and e) setting the body mark on the ultrasound image based on the edge.

This summary is provided to introduce a selection of concepts in a simplified form that are further described below in the Detailed Description. This Summary is not intended to identify key or essential features of the claimed subject matter, nor is it intended to be used in determining the scope of the claimed subject matter.

### BRIEF DESCRIPTION OF THE DRAWINGS

FIG. 1 is a block diagram showing an illustrative embodiment of an ultrasound system.

FIG. 2 is a block diagram showing an illustrative embodiment of an ultrasound data acquisition unit.

FIG. 3 is a schematic diagram showing an example of a mapping table.

FIG. 4 is a flow chart showing a process of setting a body mark on an ultrasound image.

FIG. 5 is a schematic diagram showing an example of an ultrasound image and a body mark.

### DETAILED DESCRIPTION

A detailed description may be provided with reference to the accompanying drawings. One of ordinary skill in the art may realize that the following description is illustrative only and is not in any way limiting. Other embodiments of the present invention may readily suggest themselves to such skilled persons having the benefit of this disclosure.

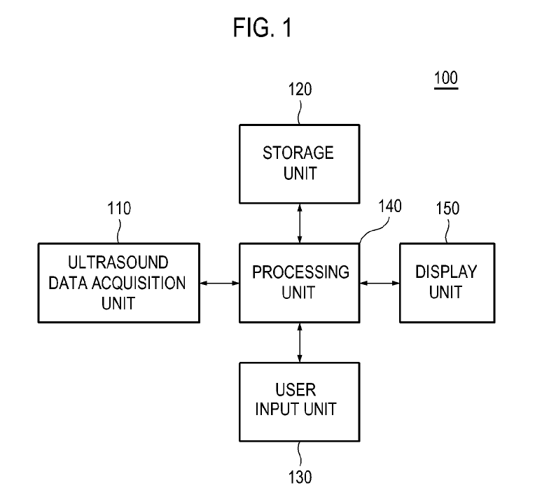

Referring to FIG. 1, an ultrasound system 100 in accordance with an illustrative embodiment is shown. As depicted therein, the ultrasound system 100 may include an ultrasound data acquisition unit 110.

The ultrasound data acquisition unit 110 may be configured to transmit ultrasound signals to a living body, which includes a target object. The target object may include a heart, a fetus and the like. The ultrasound data acquisition unit 110 may be further configured to receive ultrasound signals (i.e., ultrasound echo signals) from the living body to acquire ultrasound data.

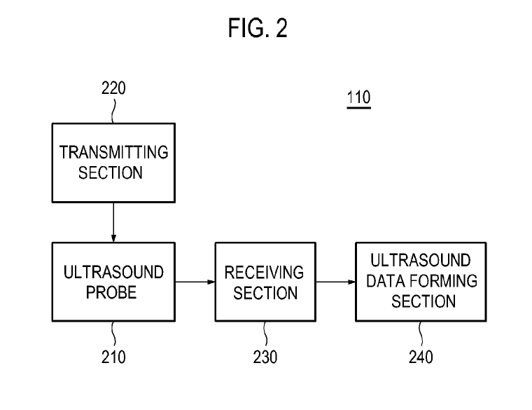

FIG. 2 is a block diagram showing an illustrative embodiment of the ultrasound data acquisition unit. Referring to FIG. 2, the ultrasound data acquisition unit 110 may include an ultrasound probe 210.

The ultrasound probe 210 may include a plurality of elements (not shown) for reciprocally converting between ultrasound signals and electrical signals. The ultrasound probe 210 may be configured to transmit ultrasound signals to the living body. The ultrasound probe 210 may be further configured to receive ultrasound echo signals from the living body to output received signals. The received signals may be analog signals. The ultrasound probe 210 may include a convex probe, a linear probe and the like.

The ultrasound data acquisition unit 110 may further include a transmitting section 220. The transmitting section 220 may be configured to control the transmission of the ultrasound signals. The transmitting section 220 may be further configured to generate electrical signals (“transmitting signals”) for obtaining an ultrasound image in consideration of the elements and focal points. Thus, the ultrasound probe 210 may be configured to convert the transmitting signals into the ultrasound signals, transmit the ultrasound signals to the living body and receive the ultrasound echo signals from the living body to thereby output the received signals. The ultrasound image may include a brightness mode image. However, it should be noted herein that the ultrasound image may not be limited thereto. The transmitting section 220 may include a transmitting signal forming section (not shown), a transmitting delay time information memory (not shown), a transmitting beam former (not shown) and the like.

The ultrasound data acquisition unit 110 may further include a receiving section 230. The receiving section 230 may be configured to convert the received signals provided from the ultrasound probe 210 into digital signals. The receiving section 230 may be further configured to apply delays to the digital signals in consideration of the elements and the focal points to thereby output digital receive-focused signals. The receiving section 230 may include an analog-to-digital converter (not shown), a receiving delay time information memory (not shown), a receiving beam former (not shown) and the like.

The ultrasound data acquisition unit 110 may further include an ultrasound data forming section 240. The ultrasound data forming section 240 may be configured to form ultrasound data corresponding to the ultrasound image based on the digital receive-focused signals. The ultrasound data may include radio frequency data. However, it should be noted herein that the ultrasound data may not be limited thereto. The ultrasound data forming section 240 may be further configured to perform signal processing (e.g., gain control, etc) upon the digital receive-focused signals.

Referring back to FIG. 1, the ultrasound system 100 may further include a storage unit 120. The storage unit 120 may store at least one body mark corresponding to at least one application. The application may represent a diagnostic part (i.e., target object) of the living body. The body mark may include a template, which represents the diagnostic part. However, it should be noted herein that the body mark may not be limited thereto.

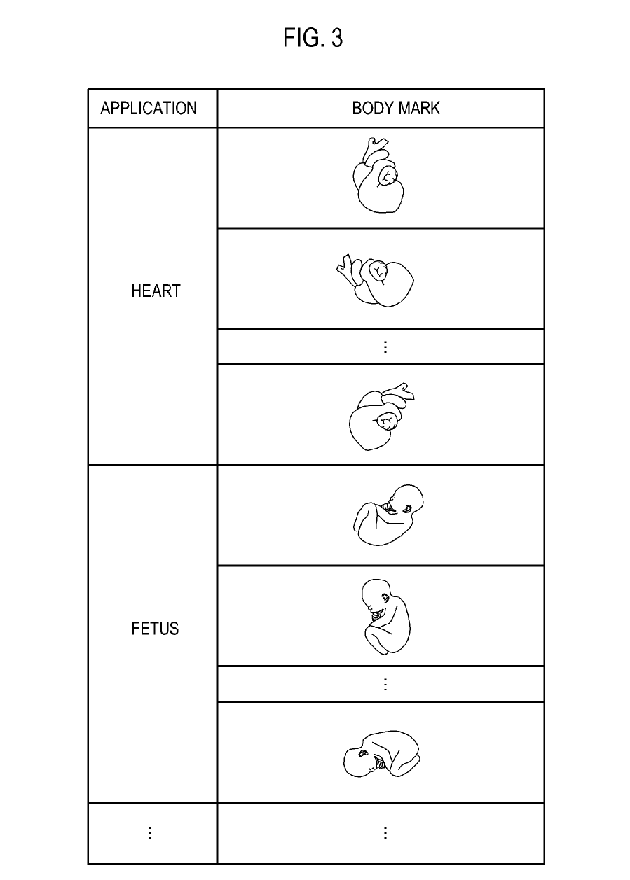

In one embodiment, the storage unit 120 may store a mapping table for providing a plurality of body marks corresponding to each of the applications (e.g., the heart, the fetus, etc.), as shown in FIG. 3. The body marks may have different shapes according to a position of the ultrasound probe 210, which is contacted on the surface of the living body. That is, the body marks may have different shapes according to a transmitting direction of the ultrasound signals.

The ultrasound system 100 may further include a user input unit 130. The user input unit 130 may be configured to receive input information from a user. In one embodiment, the input information may include first input information for selecting an application. The input information may further include second input information for adjusting the body mark. For example, the second input information may include information for adjusting a line color, a line style (e.g., a dotted line, a solid line, etc.), a line thickness, a display time (e.g., in seconds), a transparency and a position of the body mark. However, it should be noted herein that the second input information may not be limited thereto. The user input unit 130 may include a control panel, a trackball, a mouse, a keyboard and the like.

The ultrasound system 100 may further include a processing unit 140 in communication with the ultrasound data acquisition unit 110, the storage unit 120 and the user input unit 130. The processing unit 140 may include a central processing unit, a microprocessor, a graphic processing unit and the like.

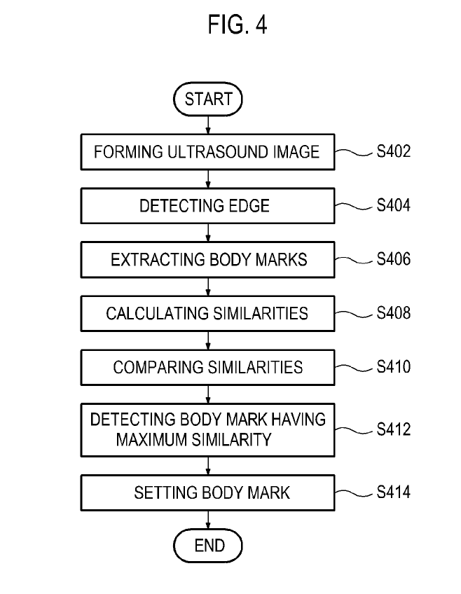

FIG. 4 is a flow chart showing a process of setting the body mark on the ultrasound image. The processing unit 140 may be configured to form the ultrasound image based on the ultrasound data provided from the ultrasound data acquisition unit 110, at step S402 in FIG. 4.

The processing unit 140 may be configured to perform edge detection upon the ultrasound image to detect an edge of the target object, at step S404 in FIG. 4. The edge may be detected by using an edge detecting mask such as a Sobel mask, Prewitt mask, Canny mask and the like. Also, the edge may be detected by using a structure tensor.

The processing unit 140 may be configured to retrieve the storage unit 120 to extract at least one body mark corresponding to the input information (i.e., first input information) provided from the user input unit 130, at step S406 in FIG. 4. The body marks may be extracted in the order stored in the storage unit 120.

The processing unit 140 may be configured to set a body mark corresponding to an application on a predetermined position of the ultrasound image, based on the detected edge and the extracted at least one body mark.

More particularly, if at least two body marks are extracted from the storage unit 120, the processing unit 140 may be configured to compare the detected edge with each of the extracted body marks to calculate similarities between the detected edge and each of the extracted body marks, at step S408 in FIG. 4. The methods of calculating the similarities are well known in the art. Thus, they have not been described in detail so as not to unnecessarily obscure the present invention. The processing unit 140 may be configured to the compare the calculated similarities, at step S410 in FIG. 4. The processing unit 140 may be configured to detect a body mark having a maximum similarity, at step S412 in FIG. 4.

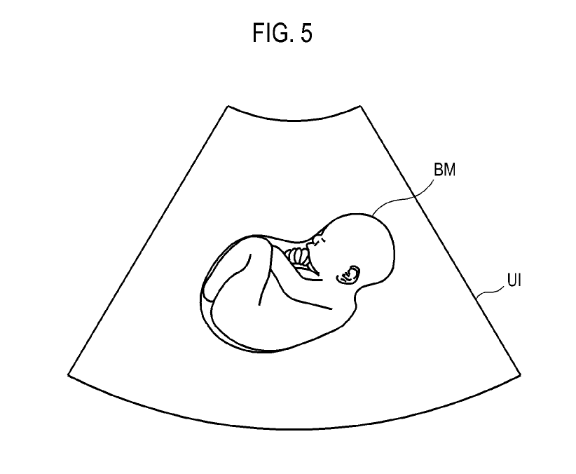

The processing unit 140 may be configured to set the detected body mark on a predetermined position of the ultrasound image, at step S414 in FIG. 4. In one embodiment, the processing unit 140 may set the detected body mark BM on the target object of the ultrasound image UI based on the detected edge, as shown in FIG. 5.

Otherwise, if one body mark is extracted from the storage unit 120, the processing unit 140 may be configured to set the extracted body mark on the target object of the ultrasound image based on the detected edge.

Optionally, the processing unit 140 may be configured to adjust the body mark set on the ultrasound image, based on the input information (i.e., second input information) provided from the user input unit 130. For example, the processing unit 140 may adjust the line color, the line style, the line thickness, the display time, the transparency and the position of the body mark set on the ultrasound image, based on the second input information.

Referring back to FIG. 1, the ultrasound system 100 may further include a display unit 150. The display unit 150 may be configured to display the ultrasound image. The display unit 150 may be further configured to display the ultrasound image and the body mark.

In another embodiment, the present invention may provide a computer readable medium comprising computer executable instructions configured to perform the following acts: a) acquiring ultrasound data corresponding to a living body including a target object; b) forming an ultrasound image based on the ultrasound data; c) performing edge detection upon the ultrasound image to detect an edge of the target object; d) detecting a body mark corresponding to the edge from a storage unit for storing at least one body mark corresponding to at least one application based on first input information for selecting an application; and e) setting the body mark on the ultrasound image based on the edge. The computer readable medium may comprise a floppy disk, hard disk, memory, compact disk, digital video disk, etc.

Although embodiments have been described with reference to a number of illustrative embodiments thereof, it should be understood that numerous other modifications and embodiments can be devised by those skilled in the art that will fall within the spirit and scope of the principles of this disclosure. More particularly, numerous variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

What is claimed is:

1. An ultrasound system, comprising:
an ultrasound data acquisition unit configured to acquire ultrasound data corresponding to a living body including a target object;
a storage unit for storing at least one body mark corresponding to at least one application; and
a processing unit configured to form an ultrasound image based on the ultrasound data, perform edge detection upon the ultrasound image to detect an edge of the target object, detect a body mark corresponding to the edge from the storage unit based on first input information for selecting an application, and set the body mark on the ultrasound image based on the edge.

2. The ultrasound system of claim 1, wherein the at least one body mark has different shapes according to a transmitting direction of ultrasound signals.

3. The ultrasound system of claim 1, wherein the processing unit is configured to:
extract at least one body mark corresponding to the first input information from the storage unit;
calculate at least one similarity between the edge and the extracted body mark; and
detect the body mark having a maximum similarity based on the calculated at least one similarity.

4. The ultrasound system of claim 1, further comprising:
a user input unit configured to receive the first input information from a user.

5. The ultrasound system of claim 4, wherein the user input unit is further configured to receive second input information for adjusting the body mark set on the ultrasound image.

6. The ultrasound system of claim 5, wherein the processing unit is further configured to adjust at least one of a line color, a line style, a line thickness, a display time, a transparency and a position of the body mark set on the ultrasound image.

7. A method of providing a body mark, comprising:
a) acquiring ultrasound data corresponding to a living body including a target object;
b) forming an ultrasound image based on the ultrasound data;
c) performing edge detection upon the ultrasound image to detect an edge of the target object;
d) detecting a body mark corresponding to the edge from a storage unit for storing at least one body mark corresponding to at least one application based on first input information for selecting an application; and
e) setting the body mark on the ultrasound image based on the edge.

8. The method of claim 7, wherein the at least one body mark has different shapes according to a transmitting direction of ultrasound signals.

9. The method of claim 7, wherein the step d) comprises:
extracting at least one body mark corresponding to the first input information from the storage unit;
calculating at least one similarity between the edge and the extracted body mark; and
detecting the body mark having a maximum similarity based on the calculated at least one similarity.

10. The method of claim 7, further comprising:
f) adjusting at least one of a line color, a line style, a line thickness, a display time, a transparency and a position of the body mark set on the ultrasound image, based on second input information for adjusting the body mark set on the ultrasound image.

11. A computer readable medium comprising computer executable instructions configured to perform following acts:
a) acquiring ultrasound data corresponding to a living body including a target object;
b) forming an ultrasound image based on the ultrasound data;
c) performing edge detection upon the ultrasound image to detect an edge of the target object;
d) detecting a body mark corresponding to the edge from a storage unit for storing at least one body mark corresponding to at least one application based on first input information for selecting an application; and
e) setting the body mark on the ultrasound image based on the edge.
